(19) United States
(12) Patent Application Publication
(10) Pub. No.: US 2020/0390324 A1
(43) Pub. Date: Dec. 17, 2020

(54) METHOD FOR TESTING EYES AND VISION TESTING SYSTEM

(71) Applicant: OCULUS OPTIKGERAETE GMBH, Wetzlar (DE)

(72) Inventor: Stephan Degle, Jena (DE)

(21) Appl. No.: 16/891,638

(22) Filed: Jun. 3, 2020

(30) Foreign Application Priority Data
Jun. 12, 2019 (DE) 10 2019 115 929.6

(51) Int. Cl. A61B 3/04; A61B 3/00; A61B 3/032; A61B 5/1171; A61B 3/028

(52) U.S. Cl. CPC A61B 3/04; A61B 3/0025; A61B 3/032; A61B 3/0285; A61B 3/0008; A61B 5/1176; A61B 3/0041

(57) ABSTRACT

A method for testing the eyes of a test person with the aid of a vision testing system as well as a vision testing system. A trial frame of the vision testing system is disposed in front of the eyes, said trial frame having at least one trial lens, at least one numerical value of an optical parameter which characterises the trial lens being recorded by means of a camera of a camera device of a display apparatus, said display apparatus being controlled by means of a control device of the vision testing system, said numerical value being identified by the control device by means of image processing, said numerical value being identified for a sphere and/or a cylinder and/or an axis and/or a prism.

This application claims priority to German Patent Application DE 10 2019 115 929.6 filed Jun. 12, 2019, which is hereby incorporated by reference in its entirety for all purposes.

The disclosure relates to a method for testing the eyes of a test person with the aid of a vision testing system as well as to a vision testing system, a trial frame of the vision testing system being disposed in front of the eyes, said trial frame having at least one trial lens.

Such vision testing systems are sufficiently known from the state of the art and are commonly used for carrying out eye tests. For instance, the known vision testing systems dispose of a display apparatus having a screen by means of which vision test symbols can be visualised or presented to a test person. Such a vision testing system can also comprise a control apparatus via which an operator is able to control a rendition of vision test symbols on the screen. Depending on the type of the screen, said screen may also dispose of a linear or circular polarisation. The polarisation of the screen is commonly utilised for carrying out eye tests in connection with a trial frame or a phoropter which is disposed in front of the eyes of the test person. Furthermore, the vision testing system can comprise a camera device having a camera which can be utilised for measuring the eyes of the test person, for example. A subjective refraction of the eyes of the test person is determined by an operator of the vision testing system presenting a variety of vision test symbols in connection with trial lenses of a trial frame or of a phoropter. Here, different eye tests which are generally known, amongst other things a test of the close vision and of the distance vision, can also be carried out with the aid of the vision testing system. Such a vision testing system is for example disclosed by EP 3 192 432 A2.

Furthermore, EP 3 178 376 A1 as well as EP 3 179 296 A1 each disclose a trial frame which serves for determining a subjective refraction of a test person and is employed in combination with a vision testing system. Said trial frame has two lens holding devices for receiving trial lenses or insert lenses, a bridge which connects the lens holding devices in a way that they are adjustable with respect to their distance from each other, a nose rest device, having a nose rest which is pivotable and adjustable in height, as well as two temples which are adjustable in length and height. Furthermore, the lens holding devices have multiple lens seats which each comprise at least one pressure finger for clamping a trial lens or insert lens. The lens seats can each be realised from at least two or three crosspieces which can retain the trial lenses or insert lenses at their circumference. Consequently, a number of trial lenses can be employed simultaneously, for correcting myopia, hyperopia and astigmatism for example.

Spherical trial lenses are employed for correcting ametropia (myopia, hyperopia) while cylindrical or prismatic trial lenses are used for correcting astigmatism (corneal irregularity) or an impairment in binocularity. According to this, each trial lens can be classified or characterised by at least one corresponding optical parameter, in particular by a sphere, a cylinder or a prism. The trial lens can then completely be characterised by indicating a numerical value of the optical parameter. Thus, a trial lens can be a spherical trial lens having a numerical value of 1.5 (dioptres) for example. In order to be able to differentiate between the trial lenses better, the trial lenses commonly include an inscription which displays the numerical value. Often, this inscription is applied to a handle of a frame of the trial lens. When testing the eyes of the test person, different trial lenses can then be inserted into the lens seats, the test person being asked for a result after each insertion procedure, until a trial lens or a combination of trial lenses is found which subjectively optimises a recognition of the vision test symbol through the eyes of the test person. Here, a disadvantage is, however, that the operator has to document all the numerical values for the inserted trial lenses by hand. This is, on the one hand, time-consuming and, on the other hand, prone to errors, in particular to errors when copying the values.

The present disclosure is therefore based on the object of proposing a method for testing the eyes of a test person with the aid of a vision testing system as well as a vision testing system by means of which a vision test can be carried out more easily.

This object is attained by a method having the features of claim 1 and a vision testing system having the features of claim 8.

In the case of the method for testing the eyes of a test person with the aid of a vision testing system, a trial frame of the vision testing system is disposed in front of the eyes, said trial frame having at least one trial lens, at least one numerical value of an optical parameter which characterises the trial lens being recorded by means of a camera of a camera device of a display apparatus of the vision testing system, said display apparatus being controlled by means of a control device of the vision testing system, said numerical value being identified by the control device by means of image processing, said numerical value being identified for a sphere and/or a cylinder and/or an axis and/or a prism.

Accordingly, the display apparatus includes a camera device having a camera by means of which a numerical value of an optical parameter which characterises the trial lens is recorded. Furthermore, the vision testing system comprises a control device which controls the display apparatus and identifies the numerical value of the optical parameter by means of image processing. Consequently, an operator does not have to write down or, in other words, document the numerical values which can be printed on the trial lenses manually anymore before or after each insertion procedure because this task is now taken over by the camera in combination with the control device. Hence, both an expenditure of time of the eye test and an error rate of the eye test can be reduced significantly. All in all, it is consequently possible to carry out the eye test even more easily.

In accordance with the disclosure, the numerical value is identified for a sphere and/or a cylinder and/or an axis and/or a prism. Hence, all the values which are relevant for an eyeglass prescription or a data card provided by an optician (called “Brillenpass”, common in Germany) are directly identified by means of image processing.

Advantageously, vision test symbols can be visualised for at least one eye of the test person by means of a screen of the display apparatus. The numerical value can be identified immediately after an insertion procedure, even before the vision test symbols are presented. Nonetheless, the identification may also be carried out during the visualisation of the vision test symbols or subsequently, before any following insertion procedure.

Furthermore, the camera can be actuated by means of at least one input element of the display apparatus. For example, the input element can be a push button, a rotary knob or a touch-sensitive surface which can be actuated by the operator or the test person after inserting the trial lenses. Before the input element is actuated, the trial frame can here be moved into a position which is favourable for an optical identification of the numerical values by the camera. This can be effected in that the test person moves his or her head, at which the trial frame may be disposed, into the corresponding position. Furthermore, it is conceivable that the camera is designed so as to be movable, for example by means of an articulated arm. Thus, it is then also possible to orient the camera in relation to the trial frame in such a manner that the numerical value can be identified in the best possible way. The input element can also be integrated into a control apparatus of the vision testing system which can be operated by remote control, for example into a mobile phone or a tablet computer. Moreover, the control apparatus can be voice-operated so that the actuation of the camera can be actuated by a spoken word. Nonetheless, it may be envisaged that the camera automatically identifies the numerical value without any additional actuation of an input element.

In a structurally advantageous design of the disclosure, the control device can store the numerical value in a database of the vision testing system. Hence, during or after the eye test, the operator can in a simple manner comprehend which trial lenses or combinations of trial lenses have been used so far or overall within the framework of the eye test. The test person can then give feedback about a change of his or her subjective visual impression after each insertion procedure. For example, the test person can say that he or she sees the vision test symbols more clearly now by means of the trial lenses which are inside a retaining unit or lens holding device of the trial frame at the moment than by means of the trial lenses which were inside the retaining unit immediately previously. Here, information on the change of the subjective visual impression is entered by means of an input element of the display apparatus and is also stored in the database by the control device. Hence, it is easy to document which trial lens or which selection of trial lenses leads or has led to an improvement or a deterioration of the subjective visual impression of the test person. All the numerical values which are relevant for an eyeglass prescription and which are connected with an optimised or the clearest subjective visual impression of the test person can be stored in the database at the end of the eye test and can be output if required, for example in the form of a printed eyeglass prescription.

A subjective refraction of the eye can also be established by means of the trial frame. Moreover, the trial frame can include colour filters or polarisation filters which are in each case adapted to a colour reproduction and/or a polarisation of the screen so that monocular or binocular eye tests can be carried out. If, for example, a trial frame having a linear or a circular polarisation is used, the display apparatus of the vision testing system can be selected in such a way that it is polarised consistently with the trial frame. A polarisation, for example with the aid of a λ/4 filter, is hence not necessary.

In a special further embodiment of the disclosure, a facial recognition of the test person can be carried out by means of the camera or another camera of the camera device. Advantageously, the facial recognition can be carried out before the actual eye test is started. The camera can then recognise if the eyes of the test person have previously already been tested with the aid of the vision testing system and open a corresponding electronic file of the test person if applicable or create a new file.

Furthermore, the vision testing system can be controlled by means of a voice-operated control apparatus of the vision testing system. Hence, the test person can tell the control apparatus by way of a spoken word, for example “better” or “worse”, if the subjective visual impression of the test person is better or worse with a selection of inserted trial lenses as compared to the previous selection of inserted trial lenses.

The disclosed vision testing system for testing the eyes of a test person comprises a display apparatus, said display apparatus comprising a screen by means of which vision test symbols can be visualised for at least one eye of the test person, a trial frame of the vision testing system being able to be disposed in front of the eyes, said trial frame having at least one trial lens, said display apparatus including a camera device having a camera by means of which at least one numerical value of an optical parameter which characterises the trial lens can be recorded, said vision testing system including a control device by means of which the display apparatus can be controlled, said numerical value being identifiable by the control device by means of image processing. With respect to the advantageous effects of the disclosed vision testing system, reference is made to the description of the advantages of the method in accordance with the disclosure.

In an advantageous version of the disclosure, the screen and the camera can be disposed in a common housing of the display apparatus. The vision testing system then also becomes particularly easily manageable since it does not require multiple apparatuses which are coupled to each other, yet spaced apart from each other, for carrying out an eye test and for adjusting glasses.

In accordance with an expedient structural design of the disclosure, the camera device can be movable into a storage position in the display apparatus or into a recording position outside of the display apparatus. Furthermore, the camera device can be disposed at the screen so that the camera can be lowered into a storage position, for example behind the screen, if required, or be moved into a recording position next to the screen for a camera recording. A movement of the camera from the storage position into the recording position and back can be effected by a drive unit of the camera device. If a comparatively large camera is used, a deflection prism may be envisaged so that the camera can be disposed behind the screen in a space-saving way.

It may also be envisaged that the screen is backlit. Here, a screen luminance of the screen can be adapted to an ambient luminance, the screen luminance being adapted proportionally as a function of the ambient luminance.

Advantageously, the display apparatus can include a stationary distance-test display apparatus whose display surface size is realised for eye tests with a distance between the eye and the display of 3 m to 10 m, preferably of 4 m to 8 m, and/or a portable close-test display apparatus whose display surface size is realised for eye tests with a distance between the eye and the display of 10 cm to 3 m, preferably 30 cm to 1 m. The distance-test display apparatus can then be utilised for representing vision test symbols for testing the distance vision and the close-test display apparatus can be utilised for representing vision test symbols for testing the close vision. The vision testing system can either include the distance-test display apparatus or the close-test display apparatus as well as other display apparatuses, if applicable, or also the distance-test display apparatus and the close-test display apparatus and other display apparatuses, if applicable. The distance-test display apparatus can preferably be positioned stationarily at the distance from the test person's eye mentioned above or be mounted to a wall. If the test person is placed at a defined distance relative to the distance-test display apparatus for carrying out eye tests, the distance between the eye and the distance-test display apparatus can precisely be determined. A display surface size of the distance-test display apparatus can then also be many times bigger than a display surface size of the close-test display apparatus since comparatively bigger vision test symbols might be displayed on the display surface of the distance-test display apparatus. Due to the fact that the close-test display apparatus can be managed portably, an operator or the test person can hold or place it at an almost arbitrary distance relative to the eyes of the test person within the distance from the eye mentioned above. Corresponding eye tests can thus be carried out at a wide variety of distances between the eye and the close-test display apparatus. Both the distance-test display apparatus and the close-test display apparatus can be operated by remote control by an operator with the aid of a control apparatus of the vision testing system. Here, a mobile phone, a tablet computer or the like can be envisaged as the control apparatus. The close-test display apparatus can also be a mobile phone, a tablet computer or the like.

In an advantageous version of the disclosure, the trial frame can be realised as a phoropter.

Advantageously, the display apparatus can include an illumination device by means of which the trial frame and/or the eye can be illuminated. In particular when eye tests are carried out under mesopic or scotopic light conditions, it is difficult, due to a reduced ambient light level, to capture eyes of a test person with the aid of a camera device for carrying out certain eye tests. It can also turn out to be difficult to record the numerical value of the optical parameter which characterises the trial lens under the light conditions mentioned above with the aid of a camera device or camera. An illumination device of the display apparatus can remedy this. It may also be envisaged to integrate the illumination device into the trial frame.

The illumination device can preferably include an infrared light source. The eyes of the test person or the trial frame, in particular the trial lens, can be illuminated with infrared light with the aid of the infrared light source regardless of the ambient lighting and be recorded by means of a camera device which is correspondingly adapted. In particular, the camera can then be an infrared camera. Hence, the eye test can then be carried out without being influenced by the ambient light level. Advantageously, the test person can in this way also be prevented from being blinded due to the eyes being illuminated with infrared light. It may also be envisaged that the illumination device comprises multiple infrared light sources, for example infrared light-emitting diodes. The infrared light source can be disposed immediately adjacent to the camera device. The infrared light source can also be disposed at the trial frame in such a manner that it directly illuminates the trial lens.

Furthermore, the trial lens can include a frame which has a handle, and which is preferably made from plastics, said frame being received by a retaining unit of the trial frame. Accordingly, a lens of the trial lens can be received in the frame and be protected by the same at a boundary of the lens. The retaining unit or lens holding device can include multiple lens seats into which a number of trial lenses or insert lenses can be introduced at the same time. The lens seats can each be realised from at least two or three crosspieces which can retain trial lenses or insert lenses at their circumference. Here, the lens seats can be realised in such a way that the crosspieces are only contacted by the frames of the trial lenses in order to protect the glass from damage such as scratches. Furthermore, the lens seats can in each instance include at least one pressure finger for clamping a trial lens. By means of the handle, the trial lens can in a simple manner be inserted into the retaining unit or be removed from the same after having been used. The trial lenses are preferably stored in a case or bag when they are not being used, the trial lenses advantageously being stored in the case in such a manner that an operator can take them out of the case by means of the handle.

In an advantageous version of the disclosure, the frame can include an inscription showing the numerical value. Here, the numerical value can be printed directly onto a material of the frame or else the frame can include a sticker or an adhesive label to which the numerical value can be applied. Here, a printing ink of the inscription can be selected in such a manner that it can be captured by the camera in the best possible way. In particular, the handle can include the inscription.

The frame can also include an RFID transponder and/or a data code. According to this, the numerical value of the optical parameter can also be encoded by means of a data code and/or with the aid of an RFID transponder. When an RFID transponder is used, additional information or data may be stored on a microchip of the RFID transponder, for example how often a trial lens has already been employed in the past. On the basis of said additional information or data, the operator can then in particular decide when a trial lens has to be replaced by a new trial lens. The frame can also include a bar code, a data matrix code or a QR code as the data code.

In accordance with a structurally advantageous design of the disclosure, the vision testing system can comprise a control apparatus for controlling the display apparatus. Here, the control apparatus can be realised as a remote control apparatus. For example, the control apparatus can be a mobile phone or a tablet computer.

The trial frame can also receive a plurality of trial lenses. This allows for correcting ametropia (myopia, hyperopia) and astigmatism and/or an impairment in binocularity at the same time.

Moreover, the trial frame can include a scale, preferably for displaying a numerical value for an axis. The axis can then be set by rotating the trial lens in a lens seat of a retaining unit of the trial frame.

A cylindrical trial lens can preferably include an opening in a frame of the trial lens, preferably in a handle of the frame. The opening can then make the scale visible to the operator or the camera so that a setting, in particular of a certain numerical value for the axis, can be made and the set numerical value can be identified by the camera. Here, a portion of the frame adjacent to the opening can include a marking which can be positioned on a point of the scale.

Other advantageous embodiments of the vision testing system result from the description of the features contained in the dependent claims which relate to method claim 1.

In the following, preferred embodiments of the disclosure are explained in more detail with reference to the enclosed drawing.

In the figures:

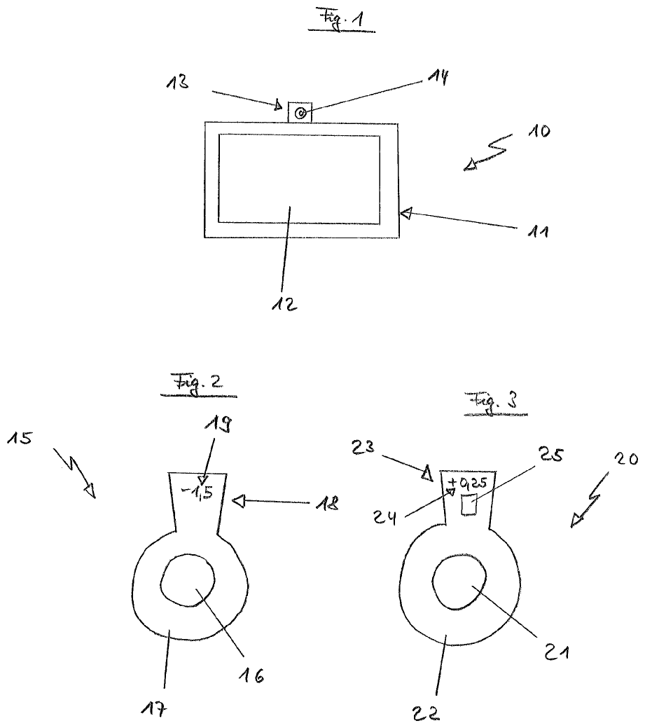

FIG. 1 shows a front view of a display apparatus;

FIG. 2 shows a front view of a trial lens in one embodiment;

FIG. 3 shows a front view of a trial lens in another embodiment.

FIG. 1 shows a vision testing system 10 having a display apparatus 11 which includes a backlit screen 12 in the manner of a television set or tablet computer. The display apparatus 11 disposes of a camera device 13 having a camera 14 by means of which at least one numerical value of an optical parameter which characterises a trial lens not shown here is recorded. Furthermore, the vision testing system 10 comprises a control device (not shown here either) by means of which the display apparatus 11 is controlled, said numerical value being identified by the control device by means of image processing. By means of the screen 12, vision test symbols are visualised for a test person in order to establish a subjective refraction value of an eye of the test person. During an eye test, a trial frame (not illustrated here) of the vision testing system 10, said trial frame having the trial lens, is disposed in front of the eyes of the test person.

FIG. 2 shows a trial lens 15 which comprises a lens 16 as well as a frame 17 having a handle 18. Furthermore, an inscription 19 is applied to the handle 18. The inscription 19 shows a numerical value “−1.5”, characterising the trial lens 15 as a spherical trial lens having −1.5 dioptres.

FIG. 3 shows a trial lens 20 which also comprises a lens 21 as well as a frame 22 having a handle 23 and an inscription 24. The inscription 24 shows a numerical value “+0.25”, characterising the trial lens 20 as a cylindrical trial lens having +0.25 dioptres. Furthermore, the handle 23 of the frame 22 includes an opening 25. The trial frame, which is not shown here, includes a scale for displaying a numerical value for an axis. If the trial lens 20 is now disposed at the trial frame, an operator can have a look at the scale through the opening 25 and set the value for the axis. In case a trial lens 15 and a trial lens 20 are disposed in front of an eye of the test person together with the trial frame, the trial lenses 15 and 20 can be rotated in such a manner that the handle 18 does not cover the opening 25, which would block the view of an operator or of the camera 14 onto the numerical value for the axis.

1. A method for testing the eyes of a test person with the aid of a vision testing system a trial frame of the vision testing system being disposed in front of the eyes, said trial frame having at least one trial lens, wherein
at least one numerical value of an optical parameter which characterises the trial lens is recorded by a camera of a camera device of a display apparatus of the vision testing system, said display apparatus being controlled by a control device of the vision testing system, said numerical value being identified by the control device by image processing, said numerical value being identified for a sphere and/or a cylinder and/or an axis and/or a prism.

2. The method according to claim 1, wherein vision test symbols are visualised for at least one eye of the test person by means of a screen of the display apparatus.

3. The method according to claim 1, wherein the camera is actuated by at least one input element of the display apparatus.

4. The method according to claim 1, wherein the control device stores the numerical value in a database of the vision testing system.

5. The method according to claim 1, wherein a subjective refraction of the eye is established by the trial frame.

6. The method according to claim 1, wherein a facial recognition of the test person is carried out by the camera or another camera of the camera device.

7. The method according to claim 1, wherein the vision testing system is controlled by a voice-operated control apparatus of the vision testing system.

8. A vision testing system for testing the eyes of a test person, said vision testing system comprising a display apparatus, said display apparatus comprising a screen by means of which vision test symbols can be visualised for at least one eye of the test person, a trial frame of the vision testing system being able to be disposed in front of the eyes, said trial frame having at least one trial lens, wherein
the display apparatus includes a camera device having a camera by means of which at least one numerical value of an optical parameter which characterises the trial lens can be recorded, said vision testing system including a control device by means of which the display apparatus can be controlled, said numerical value being identifiable by the control device by image processing.

9. The vision testing system according to claim 8, wherein the screen and the camera are disposed in a common housing of the display apparatus.

10. The vision testing system according to claim 8 wherein the camera device can be moved into a storage position in the display apparatus or into a recording position outside of the display apparatus.

11. The vision testing system according to claim 8, wherein the screen is backlit.

12. The vision testing system according to claim 8, wherein the display apparatus includes a stationary distance-test display apparatus whose display surface size is realised for eye tests with a distance between the eye and the display of 3 m to 10 m, and/or includes a portable close-test display apparatus whose display surface size is realised for eye tests with a distance between the eye and the display of 10 cm to 3 m.

13. The vision testing system according to claim 8, wherein the trial frame is realised as a phoropter.

14. The vision testing system according to claim 8, wherein the display apparatus includes an illumination device by means of which the trial frame and/or the eye can be illuminated.

15. The vision testing system according to claim 14, wherein the illumination device includes an infrared light source.

16. The vision testing system according to claim 8, wherein the trial lens includes a frame which has a handle, said frame being received by a retaining unit of the trial frame.

17. The vision testing system according to claim 16, wherein the frame includes an inscription showing the numerical value.

18. The vision testing system according to claim 16, wherein the frame includes an RFID transponder and/or a data code.

19. The vision testing system according to claim 8, wherein the vision testing system comprises a control apparatus for controlling the display apparatus.

20. The vision testing system according to claim 8, wherein the trial frame can receive a plurality of trial lenses.

21. The vision testing system according to claim 8, wherein the trial frame includes a scale, preferably for displaying a numerical value for an axis.

22. The vision testing system according to claim 21, wherein a cylindrical trial lens includes an opening in a frame of the trial lens, preferably in a handle of the frame.
